(19) United States
(12) Patent Application Publication
(10) Pub. No.: US 2020/0066380 A1
(43) Pub. Date: Feb. 27, 2020

(54) IDENTIFICATION OF AN APPROPRIATE MEDICAL INSTITUTION BASED ON PATIENT INFORMATION INCLUDING A SYMPTOM AND A MEDICAL HISTORY

(71) Applicant: FUJITSU LIMITED, Kawasaki-shi (JP)

(72) Inventors: Hiroshi Yokomichi, Yokohama (JP); Yuri Shimada, Yokohama (JP)

(73) Assignee: FUJITSU LIMITED, Kawasaki-shi (JP)

(21) Appl. No.: 16/539,493

(22) Filed: Aug. 13, 2019

(30) Foreign Application Priority Data
Aug. 24, 2018 (JP) 2018-157070

(51) Int. Cl. G16H 10/60; G16H 50/20; G16H 50/70

(52) U.S. Cl. CPC G16H 10/60; G16H 20/10; G16H 50/70; G16H 50/20

(57) ABSTRACT

An apparatus acquires, from a patient information storage device that stores patient information including a symptom of each of a plurality of patients and a medical history of each of the plurality of patients, the patient information of each of the plurality of patients. The apparatus extracts first patient information including a symptom of a target patient from the patient information of each of the plurality of patients, estimates a first disease corresponding to the symptom of the target patient based on a medical history included in the first patient information. The apparatus searches for first medical institution information including the first disease, from medical institution information including identification information indicating each of a plurality of medical institutions and a disease that has been treated at each of the plurality of medical institutions, and outputs first identification information included in the first medical institution information including the first disease.

### CROSS-REFERENCE TO RELATED APPLICATION

This application is based upon and claims the benefit of priority of the prior Japanese Patent Application No. 2018-157070, filed on Aug. 24, 2018, the entire contents of which are incorporated herein by reference.

### FIELD

The embodiments discussed herein are related to identification of an appropriate medical institution based on patient information including a symptom and a medical history.

### BACKGROUND

In recent years, the need for a big data analysis is increasing. In the big data analysis, it is desirable to collect as many data samples as possible to obtain a more accurate and useful analysis result.

The government plans to create a system of collecting medical information such as electronic medical records from medical institutions across the country to use the medical information as big data in order to promote a big data analysis in the medical field in the country. Medical information collected from medical institutions throughout the country is not only utilized in big data analysis, but also is greatly useful for a patient in selecting a medical institution from which the patient is to receive a medical examination.

An information processing apparatus is known which reduces the risk of information leakage when medical information collected from medical institutions throughout the country is analyzed regarding medical information (see, for example, Japanese Laid-open Patent Publication No. 2018-28886). There is also known a method of providing emergency medical information, an electronic medical record management program for deterring browsing of an electronic medical record, which is a violation of the privacy of a patient, and an information processing apparatus for deriving an appropriate medicine dosage (for example, see Japanese Laid-open Patent Publication No. 2012-212199, Japanese Laid-open Patent Publication No. 2017-111665, and International Publication Pamphlet No. WO 2017/146067). There is also a search site for searching for a hospital on the Internet (see, for example, <URL: https://hospia.jp/Home/Toplst?id=0>, “Hospital Intelligence Agency”, Internet online searched on Jun. 25, 2018).

### SUMMARY

According to an aspect of the embodiments, an apparatus acquires, from a patient information storage device that stores patient information including a symptom of each of a plurality of patients and a medical history of each of the plurality of patients, the patient information of each of the plurality of patients. The apparatus extracts first patient information including a symptom of a target patient from the patient information of each of the plurality of patients, estimates a first disease corresponding to the symptom of the target patient based on a medical history included in the first patient information. The apparatus searches for first medical institution information including the first disease, from medical institution information including identification information indicating each of a plurality of medical institutions and a disease that has been treated at each of the plurality of medical institutions, and outputs first identification information included in the first medical institution information including the first disease.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a functional block diagram of an information processing apparatus;

FIG. 2 is a flowchart of medical institution search processing;

FIG. 3 is a block diagram of a medical institution search system;

FIG. 4 is a diagram illustrating diagnosis and treatment information;

FIG. 5 is a diagram illustrating purchase information;

FIG. 6 is a diagram illustrating login information;

FIG. 7 is a diagram illustrating a search request;

FIG. 8 is a diagram illustrating a comparison target list;

FIG. 9 is a diagram illustrating a narrowing-down result;

FIG. 10 is a diagram illustrating a patient list;

FIG. 11 is a diagram illustrating medical institution information;

FIG. 12 is a diagram illustrating a search result;

FIG. 13 is a diagram illustrating a login screen;

FIG. 14 is a diagram illustrating a search screen;

FIG. 15 is a diagram illustrating an information collection sequence;

FIG. 16A is a diagram illustrating a medical institution search sequence (part 1);

FIG. 16B is a diagram illustrating a medical institution search sequence (part 2); and

FIG. 17 is a block diagram of hardware of the information processing apparatus.

### DESCRIPTION OF EMBODIMENTS

When a patient who suffers from an illness searches for a hospital using a search site on the Internet, the patient judges the symptom by oneself, searches for an organization dealing with information such as hospitals on the Internet, and determines by oneself the hospital and its diagnosis and treatment department from which the patient receives a medical examination.

However, it is difficult to find an appropriate hospital that matches the symptom of the patient if the patient determines the symptom by oneself.

Such a problem arises not only when a patient searches for a hospital but also when the patient's family, medical staff, and the like search for a hospital.

It is preferable to search for a medical institution based on the symptom of a patient to identify the appropriate medical institution.

Hereinafter, embodiments will be described in detail with reference to the drawings.

In the information processing system of Japanese Laid-open Patent Publication No. 2018-28886, a cloud environment is constructed in a data center, and medical record information of each hospital is collected. The patient's personal information included in the medical record information is classified, and the classified medical record information is accumulated in the database. As a result, when searching for the accumulated medical record information, it is possible to refer to the medical record information while concealing the information identifying the individual.

In this information processing system, only an information analysis institution may refer to unclassified medical record information and personal information of the patient, and an information use institution such as a research institute and a pharmaceutical company may refer to the only analysis result of medical record information not including personal information.

Even in the information analysis institution, an access to the medical record information not classified by the analyst is prohibited by separating the first communication network through which unclassified medical record information is transmitted and the second communication network accessible to the analyst. In this case, the first communication network and the second communication network are respectively connected to different network interfaces of the storage apparatus that stores the classified medical record information, and are physically separated. Therefore, the security of unclassified medical record information is improved, and the risk of leakage of patient's personal information through the analyst is reduced.

On the other hand, when a patient who has suffered from an illness searches for a hospital using a search site on the Internet, the patient judges the symptom by oneself, searches for an organization dealing with information such as hospitals on the Internet, and determines by oneself the hospital and its diagnosis and treatment department from which the patient receives a medical examination.

However, it is difficult to find an appropriate hospital that matches the symptom of the patient if the patient determines the symptom by oneself. For example, if the symptom is fever, the patient will often search for the hospital judging that the cause is a cold, but will not perform the search by taking into consideration the possibility of other infections.

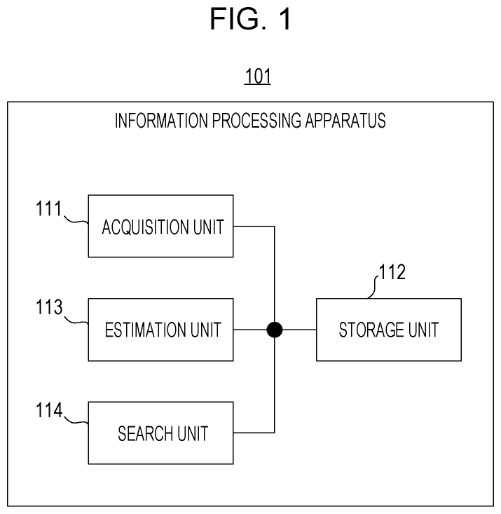

FIG. 1 illustrates an example of the functional configuration of the information processing apparatus (computer) of the embodiment. An information processing apparatus 101 in FIG. 1 includes an acquisition unit 111, a storage unit 112, an estimation unit 113, and a search unit 114.

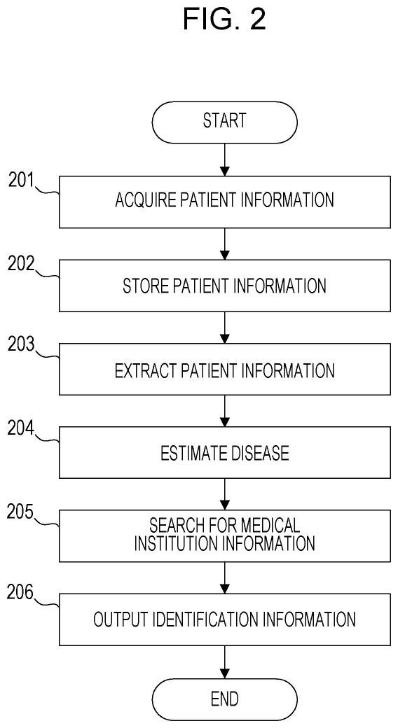

FIG. 2 is a flowchart illustrating an example of medical institution search processing performed by the information processing apparatus 101 of FIG. 1. First, the acquisition unit 111 acquires patient information of each of a plurality of patients from a patient information storage device that stores patient information including the symptom of each of a plurality of patients and the medical history of each of the patients (step 201), and stores the acquired patient information in the storage unit 112 (step 202).

Next, the estimation unit 113 extracts patient information including the symptom of the target patient from among the patient information stored in the storage unit 112 (step 203), and estimates a disease corresponding to the symptom of the target patient based on the medical history included in the extracted patient information (step 204).

Next, the search unit 114 searches for medical institution information including the disease estimated by the estimation unit 113 out of medical institution information including identification information indicating each of a plurality of medical institutions and a disease which has been treated at each of the medical institutions (step 205). The search unit 114 outputs the identification information included in the medical institution information including the disease estimated by the estimation unit 113 (step 206).

The information processing apparatus 101 of FIG. 1 makes it possible to search for a medical institution based on the symptom of a patient to identify the appropriate medical institution. For example, examples of medical institutions include hospitals, clinics, and nursing homes for the elderly.

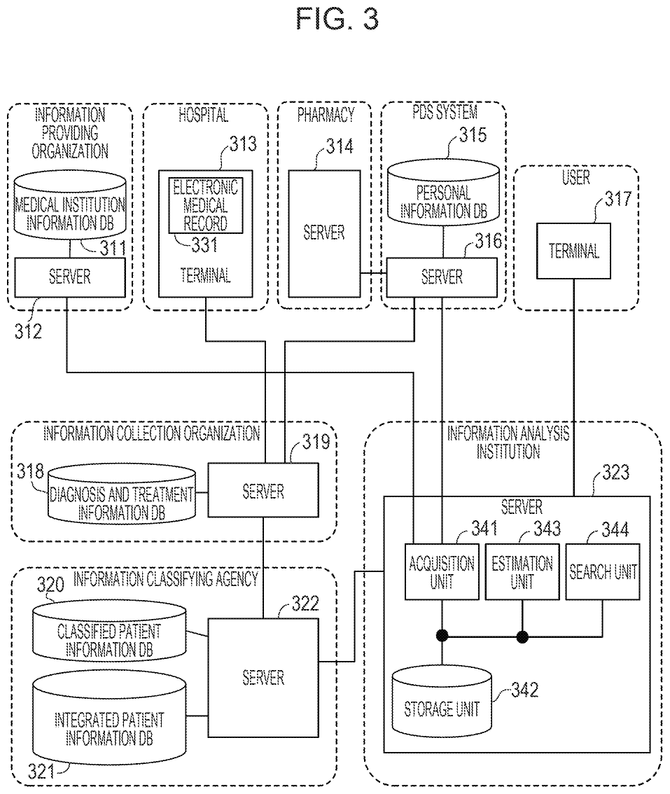

FIG. 3 illustrates an example of a configuration of a medical institution search system including the information processing apparatus 101 of FIG. 1. The medical institution search system of FIG. 3 includes a medical institution information database (DB) 311, a server 312, a terminal 313, a server 314, a personal information DB 315, a server 316, and a terminal 317. The medical institution search system includes a diagnosis and treatment information DB 318, a server 319, a classified patient information DB 320, an integrated patient information DB 321, a server 322, and a server 323.

The medical institution information DB 311 is a storage device of an information providing organization, and stores medical institution information. The server 312 is an information processing apparatus of an information providing organization. The information providing organization is an organization providing medical institution information, and may be a government or a private organization.

The terminal 313 is an information processing apparatus of a hospital, and stores an electronic medical record 331 which is diagnosis and treatment information of a patient. The terminal 313 is provided, for example, in each of a plurality of hospitals throughout the country.

The server 314 is an information processing apparatus of a pharmacy, and stores, of the medicine sold in the pharmacy, sales information including information of a medicine name, a sales date, a purchaser, and the like. The server 314 is provided, for example, in each of a plurality of pharmacies throughout the country.

The personal information DB 315 is a personal information storage device of a personal data store (PDS) system, and stores personal information of the user. The PDS system enables a user to accumulate and manage all personal information of his or her own, and to utilize the personal information in the service of a source the access right to which is given. The personal information of the user includes diagnosis and treatment information of the user in any of the hospitals, purchase information indicating a medicine purchased by the user, and the like. The server 316 is an information processing apparatus of the PDS system. The PDS system is provided, for example, for each user.

The terminal 317 is an information processing apparatus such as a smartphone, a tablet, or a personal computer used by a user who is a target patient.

The diagnosis and treatment information DB 318 is a storage device of an information collection organization, and stores diagnosis and treatment information of each patient. The server 319 is an information processing apparatus of an information collection organization. The information collection organization is an organization that collects and stores diagnosis and treatment information from each hospital. The diagnosis and treatment information DB 318 is provided, for example, for each patient.

The classified patient information DB 320 and the integrated patient information DB 321 are patient information storage devices of the information classifying agency. The classified patient information DB 320 stores the classified patient information of each patient, and the integrated patient information DB 321 stores the classified patient information of a plurality of patients as integrated patient information. The integrated patient information corresponds to big data including a large pieces of patient information. The server 322 is an information processing apparatus of an information classifying agency. The information classifying agency is an organization that classifies and stores diagnosis and treatment information of patients. The classified patient information DB 320 is provided, for example, for each patient.

The server 323 is an information processing apparatus of an information analysis institution, and includes an acquisition unit 341, a storage unit 342, an estimation unit 343, and a search unit 344. The information analysis institution performs medical institution search processing based on the user's symptom and identifies a hospital that is adapted to the symptom. The server 323 corresponds to the information processing apparatus 101 in FIG. 1, and the acquisition unit 341, the storage unit 342, the estimation unit 343, and the search unit 344 correspond to the acquisition unit 111, the storage unit 112, the estimation unit 113, and the search unit 114, respectively.

The server 312, the server 316, and the server 322 communicate with the server 323 via a communication network, and the terminal 313, the server 316, and the server 322 communicate with the server 319 via a communication network. The server 314 communicates with the server 316 via a communication network, and the terminal 317 communicates with the server 323 via a communication network.

The medical institution search system of FIG. 3 collects, for example, the diagnosis and treatment information of the user in the following procedure.

(P1) The user visits a hospital and sees a doctor as a patient.

(P2) The doctor inputs the medical examination result to the terminal 313, and the terminal 313 generates the electronic medical record 331.

(P3) The terminal 313 transmits the electronic medical record 331 to the server 319. The server 319 stores the received electronic medical record 331 in the diagnosis and treatment information DB 318 as diagnosis and treatment information of the user. As a result, the diagnosis and treatment information of the user is backed up in the diagnosis and treatment information DB 318. When the user has a medical examination at a plurality of hospitals, diagnosis and treatment information is collected from the terminal 313 of each hospital and stored in the diagnosis and treatment information DB 318. Communication between the terminal 313 and the server 319 is encrypted.

(P4) The server 319 performs a synchronization process, extracts predetermined information from the diagnosis and treatment information of the diagnosis and treatment information DB 318, and transmits the extracted information to the server 316. The server 316 stores the received information in the personal information DB 315 as diagnosis and treatment information of the user. Communication between the server 319 and the server 316 is encrypted.

The server 319 may perform the synchronization process at the timing when the electronic medical record 331 of the user is updated, or may perform the synchronization process periodically, such as once a month. The medical institution search system may also collect diagnosis and treatment information of the user, for example, using the technology described in Japanese Patent Application No. 2017-81245, which is the prior application.

The server 319 transmits the diagnosis and treatment information of the diagnosis and treatment information DB 318 to the server 322 at the timing of performing the synchronization process. The server 322 classifies the personal information of the user included in the received diagnosis and treatment information to generate the classified patient information of the user to store the classified patient information in the classified patient information DB 320. Communication between the server 319 and the server 322 is encrypted. The server 322 may generate classified patient information using, for example, the technology described in Japanese Laid-open Patent Publication No. 2018-28886.

The server 322 stores the classified patient information of the plurality of users in the integrated patient information DB 321 as integrated patient information.

The medical institution search system of FIG. 3 collects, for example, the purchase information of the user in the following procedure.

(P11) The user purchases a medicine at a pharmacy. The medicine to be purchased may be prescribed by a doctor of the hospital from which the user has received a medical examination, or may be selected by the user by oneself.

(P12) The pharmacy salesperson inputs sales information of a medicine into the server 314.

(P13) The server 314 performs a synchronization process, extracts predetermined information from the sales information, and transmits the extracted information to the server 316. The server 316 stores the received information in the personal information DB 315 as purchase information of the user. Communication between the server 314 and the server 316 is encrypted.

The server 314 may perform the synchronization process at the timing when the user purchases the medicine, or may perform the synchronization process periodically, such as once a month. The medical institution search system may also collect purchase information of the user, for example, using the technology described in Japanese Patent Application No. 2017-199730, which is the prior application.

In the medical institution search system of FIG. 3, when a user A, the target patient, has suffered from an illness and a specific symptom occurs, medical institution search processing is performed in the following procedure, for example.

(P21) The user A logs in to a medical institution search service provided by an information analysis institution using a patient application program operating in the terminal 317, and inputs a search request to the terminal 317. The search request includes a user ID, which is identification information of the user A, and a symptom which the user A is aware of.

(P22) The terminal 317 transmits the search request to the server 323, and the acquisition unit 341 of the server 323 extracts the user ID from the received search request.

(P23) The acquisition unit 341 transmits a personal information request including the extracted user ID to the server 316.

(P24) The server 316 extracts the user ID from the received personal information request, and acquires the personal information of the user A corresponding to the extracted user ID from the personal information DB 315. The server 316 transmits the acquired personal information to the server 323. The personal information of user A includes diagnosis and treatment information in the hospital from which the user A received a medical examination before the current symptom occurs and purchase information indicating a medicine purchased by the user A before the current symptom occurs. Communication between the server 316 and the server 323 is encrypted.

(P25) The acquisition unit 341 transmits the patient information request to the server 322 after receiving authentication of a patient information providing service provided by the information classifying agency.

(P26) The server 322 acquires integrated patient information from the integrated patient information DB 321, and transmits the acquired integrated patient information to the server 323. The classified patient information included in the integrated patient information includes information of a plurality of items. Those items include a symptom, a medical history, and a medication history of the patient. Communication between the server 322 and the server 323 is encrypted.

(P27) The acquisition unit 341 stores, in the storage unit 342, the personal information received from the server 316 and the integrated patient information received from the server 322 via a virtual machine (VM) of the user A operating in the server 323.

(P28) The estimation unit 343 extracts the patient information including the symptom included in the search request from the integrated patient information by comparing the search request and the personal information with the integrated patient information. The estimation unit 343 estimates a disease corresponding to the symptom of the user A based on the medical history included in the extracted patient information.

(P29) The acquisition unit 341 transmits a medical institution information request to the server 312.

(P30) The server 312 acquires medical institution information from the medical institution information DB 311, and transmits the acquired medical institution information to the server 323. The medical institution information includes a hospital name, which is identification information of each of a plurality of medical institutions, and a disease name indicating a disease that has been treated at the hospital.

(P31) The acquisition unit 341 stores the medical institution information received from the server 312 in the storage unit 342.

(P32) The search unit 344 searches for the medical institution information for medical institution information including the disease estimated by the estimation unit 343, and outputs the hospital name included in the medical institution information as a search result.

(P33) The search unit 344 transmits the search result to the terminal 317, and the terminal 317 displays the received search result on a screen.

The medical institution search system of FIG. 3 may accurately estimate the user's disease by using the past diagnosis and treatment information and purchase information of the user and the integrated patient information, which is big data, in addition to the search request inputted by the user. This makes it possible to automatically identify an appropriate medical institution from the estimated disease to present it to the user.

Instead of the user, a family member of the user or a medical staff member such as a rescue worker may log in to the medical institution search service from another terminal to acquire the search result.

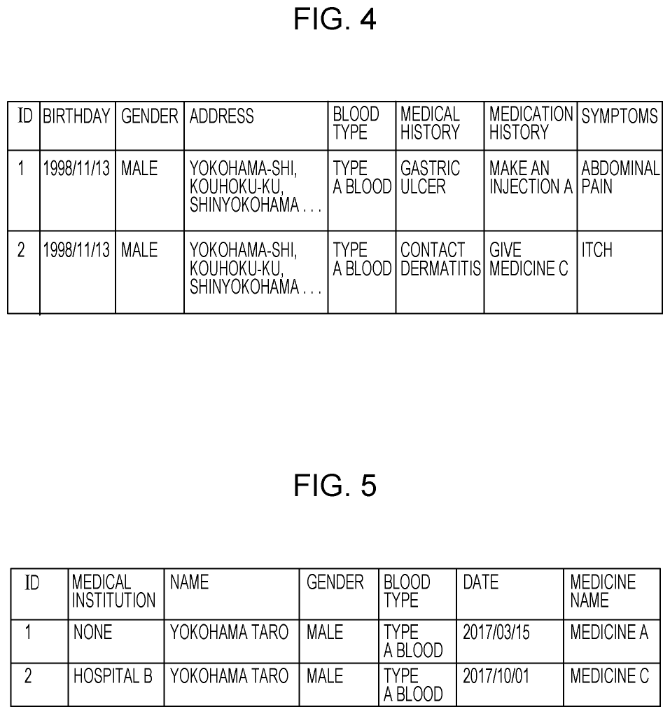

FIG. 4 illustrates an example of diagnosis and treatment information stored in the personal information DB 315. The diagnosis and treatment information in FIG. 4 includes an ID, a date of birth, a gender, an address, a blood type, a medical history, a medication history, and a symptom. The ID is identification information of entry of the diagnosis and treatment information, the medical history represents the name of the disease identified by the medical examination, the medication history represents the medicine used for the treatment, and the symptom represents the symptom identified by the medical examination.

FIG. 5 illustrates an example of purchase information stored in the personal information DB 315. The purchase information in FIG. 5 includes an ID, a medical institution, a name, a gender, an address, a blood type, a date, and a medicine name. The ID is the identification information of entry of the purchase information, the medical institution represents the name of the hospital that prescribed the medicine, the date represents the date when the user purchased the medicine, and the medicine name represents the medicine purchased by the user. The medicine A of the ID “1” is a commercially available medicine that the user has selected and purchased by oneself, and the medicine C of the ID “2” is a medicine prescribed by a doctor of the hospital B.

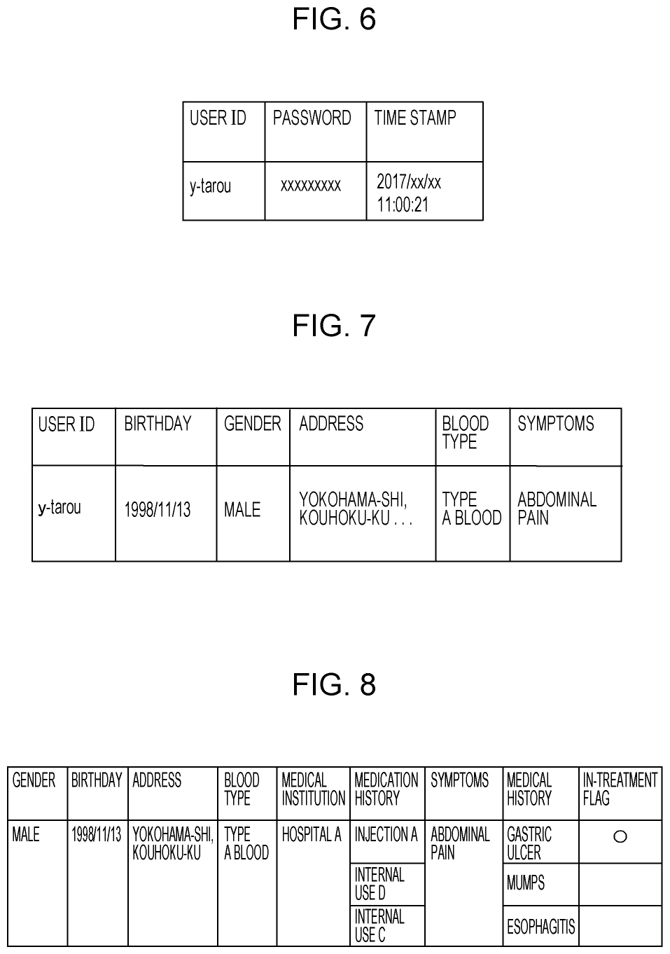

FIG. 6 illustrates an example of login information acquired by the server 323 when the user logs in to the medical institution search service in procedure (P21). The login information of FIG. 6 includes a user ID, a password, and a time stamp. The user ID and the password are input to the terminal 317 by the user.

FIG. 7 illustrates an example of a search request input by the user in procedure (P21). The search request of FIG. 7 includes a user ID, a date of birth, a gender, an address, a blood type, and a symptom. The symptom represents a symptom that the user has judged by oneself.

FIG. 8 illustrates an example of the comparison target list generated from the search request and the diagnosis and treatment information and purchase information included in the personal information in procedure (P28). The estimation unit 343 generates a comparison target list by extracting and combining information of predetermined items from the search request, the diagnosis and treatment information, and the purchase information to store the comparison target list in the storage unit 342.

The comparison target list of FIG. 8 includes a gender, a date of birth, an address, a blood type, a medical institution, a medication history, a symptom, a medical history, and an in-treatment flag. The gender, the date of birth, the address, the blood type, and the symptom are information extracted from the search request, the medical institution is information extracted from the purchase information, and the medical history is information extracted from the diagnosis and treatment information. The in-treatment flag indicates whether a disease corresponding to a medical history is currently being treated.

The medication history is information extracted from the diagnosis and treatment information and the purchase information. For example, the injection A and the medicine C described in the medication history of diagnosis and treatment information in FIG. 4 are described in the medication history in the comparison target list. The medicine A and the medicine C described in the medicine name of the purchase information in FIG. 5 are also described in the medication history in the comparison target list.

The estimation unit 343 narrows down integrated patient information to generate a narrowing-down result by comparing a predetermined item in the comparison target list with the same item of integrated patient information. For example, the estimation unit 343 may narrow down the integrated patient information according to the following procedure.

(P41) The estimation unit 343 extracts patient information including the symptom same as the symptom in the comparison target list from the integrated patient information.

(P42) The estimation unit 343 extracts patient information including the medical history same as any of the medical histories in the comparison target list from the extracted patient information. In this case, the estimation unit 343 may extract patient information in descending order, starting from patient information including a largest number of the same medical histories.

(P43) The estimation unit 343 extracts patient information including the medication history same as any of the medication histories in the comparison target list from the extracted patient information. In this case, the estimation unit 343 may extract patient information in descending order, starting from patient information including a largest number of the same medication histories.

(P44) The estimation unit 343 extracts patient information including the gender same as the gender in the comparison target list from the extracted patient information.

(P45) The estimation unit 343 extracts patient information including the blood type same as the blood type in the comparison target list from the extracted patient information.

By extracting patient information including the symptom in the comparison target list from the integrated patient information, it is possible to narrow down the patient information by using, as a key, the symptom of the user A who is the target patient. This makes it possible to estimate the disease of the user A based on the medical history of the extracted patient information.

By extracting the patient information including the medical history of the comparison target list from the integrated patient information, the patient information may be narrowed down using, as a key, the medical history of the user A. By extracting the patient information including the medication history of the comparison target list from the integrated patient information, the patient information may be narrowed down using, as a key, the medication history of the user A. By narrowing down the patient information with the medical history or the medication history as a key, the patient information of a patient whose constitution is similar to that of the user A is extracted, so that the estimation accuracy of the disease is improved.

By extracting the patient information including the gender of the comparison target list from the integrated patient information, the patient information may be narrowed down using, as a key, the gender of the user A. By extracting the patient information including the blood type of the comparison target list from the integrated patient information, the patient information may be narrowed down using, as a key, the blood type of the user A. By narrowing down the patient information with the gender or the blood type as a key, the patient information of a patient whose constitution is further similar to that of the user A is extracted, so that the estimation accuracy of the disease is improved.

The estimation unit 343 may narrow down patient information by using, as a key, another item such as the date of birth, and the address, and may omit some or all of the processes of procedure (P42) to procedure (P45).

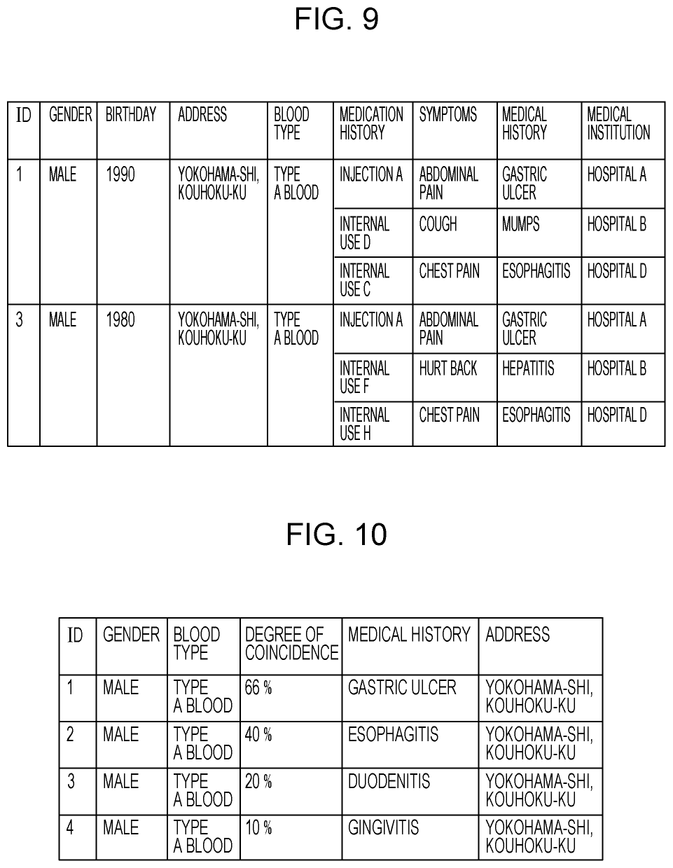

FIG. 9 illustrates an example of the narrowing-down result. The narrowing-down result in FIG. 9 includes an ID, a gender, a date of birth, an address, a blood type, a medication history, a symptom, a medical history, and a medical institution, which are the items of integrated patient information. The ID is identification information of entry of the integrated patient information, and is assigned for each patient information. The items other than the ID correspond to the items of the diagnosis and treatment information stored in the diagnosis and treatment information DB 318, and the date of birth and the address are converted into information in which the individual is not identified.

The medical institution represents the name of the hospital from which the patient has received a medical examination, the symptom and the medical history represent the symptom and the disease name identified by the medical examination, and the medication history represents the medicine used for the treatment. The medication history, the symptom, the medical history, and the medical institution of each piece of patient information include a plurality of data, and the data of the medication history, the symptom, the medical history, and the medical institution are associated with one another.

Next, the estimation unit 343 generates a patient list of patients having a medical history corresponding to the symptom of the user A from the comparison target list and the narrowing-down result to store the patient list in the storage unit 342.

FIG. 10 illustrates an example of a patient list. The patient list in FIG. 10 includes an ID, a gender, a blood type, a degree of coincidence, a medical history, and an address. The ID is identification information of entry of the patient list, and the gender, the blood type, the medical history, and the address are information extracted from the patient information included in the narrowing-down result. The medical history corresponds to the estimation result of the disease of the user A, and represents the medical history associated with the symptom same as the symptom of the user A among the medical histories included in each patient information of the narrowing-down result.

The degree of coincidence indicates the degree to which the information of the plurality of items included in the comparison target list and the information of the plurality of items included in each patient information of the narrowing-down result match, and is obtained by the following Equation.

Degree of coincidence=(M/N)×100 (1)

where N represents the number of items included in the comparison target list, and M represents the number of items having the information same as that of the comparison target list among the items included in each patient information of the narrowing-down result in Expression (1). The estimation unit 343 obtains the degree of coincidence of each piece of patient information using Expression (1), and records the obtained degree of coincidence in the patient list.

When the medication history of each piece of patient information includes any of the medicines described in the medication history in the comparison target list, it is determined that the medication history of the patient information has the information same as that in the comparison target list. When the symptom of each piece of patient information includes the symptom in the comparison target list, it is determined that the symptom of the patient information has the information same as that in the comparison target list. When the medical history of each piece of patient information includes any of the disease names described in the medical history in the comparison target list, it is determined that the medical history of the patient information has the information same as that in the comparison target list.

For example, in the case in the comparison target list in FIG. 8, N=9 and in the case of patient information indicated by the ID “1” in FIG. 9, the gender, the address, the blood type, the medication history, the symptom, and the medical history have the information same as that in the comparison target list, so that M=6. Therefore, according to Equation (1), the degree of coincidence of the patient information is 66%. The entries in the patient list of FIG. 10 are sorted in descending order of the degree of coincidence. Among these entries, the entry indicated by the ID “1” corresponds to the patient information indicated by the ID “1” in FIG. 9.

In procedure (P32), the search unit 344 extracts medical institution information including each medical history of the patient list from medical institution information to generate a search result using the extracted medical institution information.

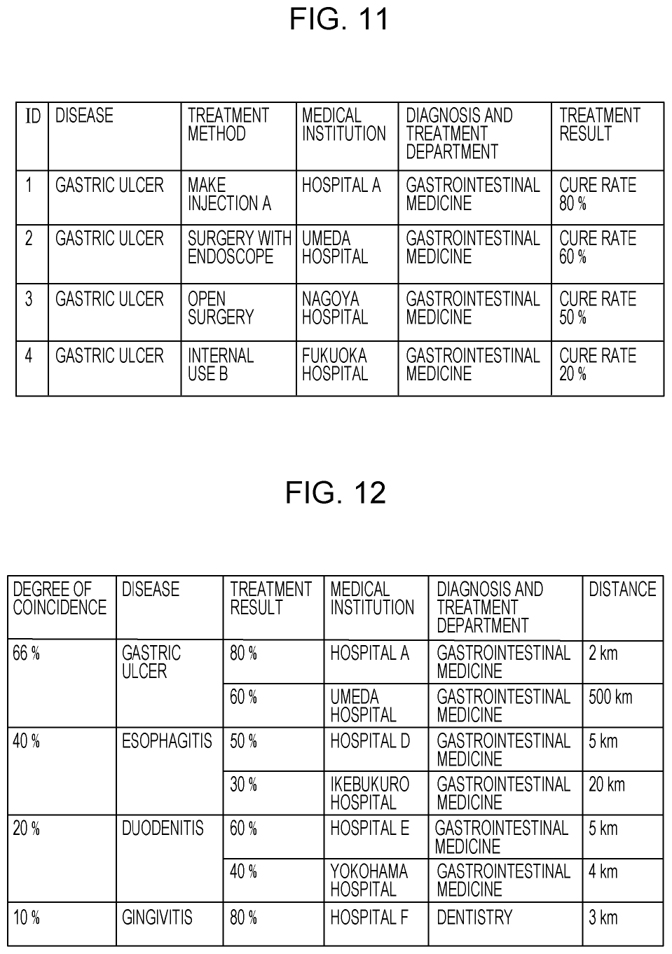

FIG. 11 illustrates an example of medical institution information including “gastric ulcer” described as the medical history of the ID “1” in FIG. 10. The medical institution information of FIG. 11 includes an ID, a disease, a treatment method, a medical institution, a diagnosis and treatment department, and a treatment result. The ID is identification information of entry of the medical institution information, and the disease represents a disease that has been treated at the medical institution. In this example, the disease is “gastric ulcer”. The treatment method represents a method used to treat the disease, the medical institution represents a hospital name, the diagnosis and treatment department represents a diagnosis and treatment department to which the doctor who perform the treatment belongs, and the treatment result represents a cure rate of the disease in the medical institution.

For example, the search unit 344 extracts a predetermined number of entries in descending order of the cure rate among medical institution information including the disease same as that of the medical history in the patient list to generate a search result including the extracted entries. In the case of extracting two entries from the medical institution information of FIG. 11, the entries of the ID “1” and the ID “2” are extracted.

FIG. 12 illustrates an example of a search result generated using the patient list of FIG. 11. The search result in FIG. 12 include a degree of coincidence, a disease, a treatment result, a medical institution, a diagnosis and treatment department, and a distance. The degree of coincidence is information extracted from the patient list, and the disease, the treatment result, the medical institution, and the diagnosis and treatment department are information extracted from medical institution information. The distance represents the distance from the user A's home to the medical institution calculated by the search unit 344 using the address of the user A.

When the search result of FIG. 12 is displayed on the screen of the terminal 317, for example, the user A may determine that the disease associated with the maximum degree of coincidence is a disease corresponding to his/her symptom. The user A selects, as the medical institution and the diagnosis and treatment department from which the user A is to receive a medical examination, the medical institution and the diagnosis and treatment department having the highest treatment result among the medical institutions and diagnosis and treatment departments associated with the disease. The user A may also select, as the medical institution and the diagnosis and treatment department from which the user A is to receive a medical examination, the medical institution and the diagnosis and treatment department having the smallest distance from the address of the user A.

In the example of FIG. 12, the gastric ulcer associated with the degree of coincidence having the maximum value of 66% is regarded as the disease of the user A, and among Hospital A and Umeda Hospital associated with the gastric ulcer, Hospital A, which has a higher treatment result and has a smaller distance, is selected as the medical institution from which the user A is to receive a medical examination. In this case, the diagnosis and treatment department from which the user A is to receive a medical examination is the digestive internal medicine department.

Thus, by displaying the search result in which the disease name, the hospital name, and the degree of coincidence are associated with each other, the user A judges that the disease having a large degree of coincidence is his or her own disease to receive a medical examination from the hospital which is suitable for the treatment of the disease.

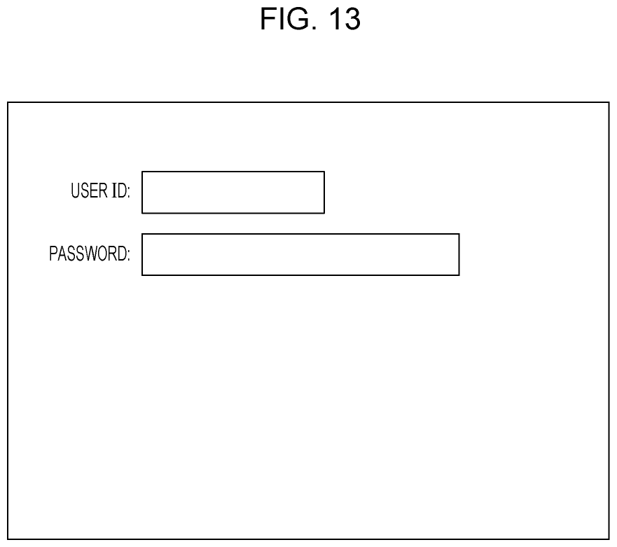

FIG. 13 illustrates an example of a login screen displayed by the terminal 317 in procedure (P21). The login screen of FIG. 13 includes a text box for inputting a user ID and a password.

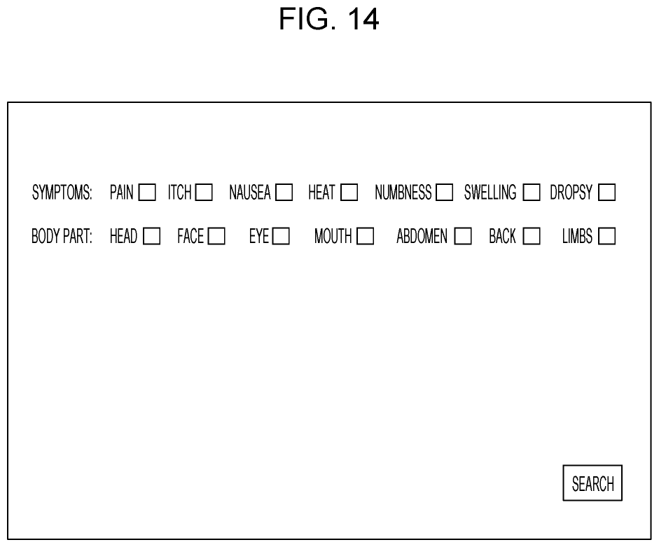

FIG. 14 illustrates an example of a search screen displayed by the terminal 317 in procedure (P21). The search screen of FIG. 14 includes a plurality of check boxes for specifying a symptom and a plurality of check boxes for specifying a body part. The user A may input the noticed symptom as a search request by selecting the combination of the symptom and the body part. For example, when a combination of “pain” and “abdomen” is selected, a symptom of “abdominal pain” is input.

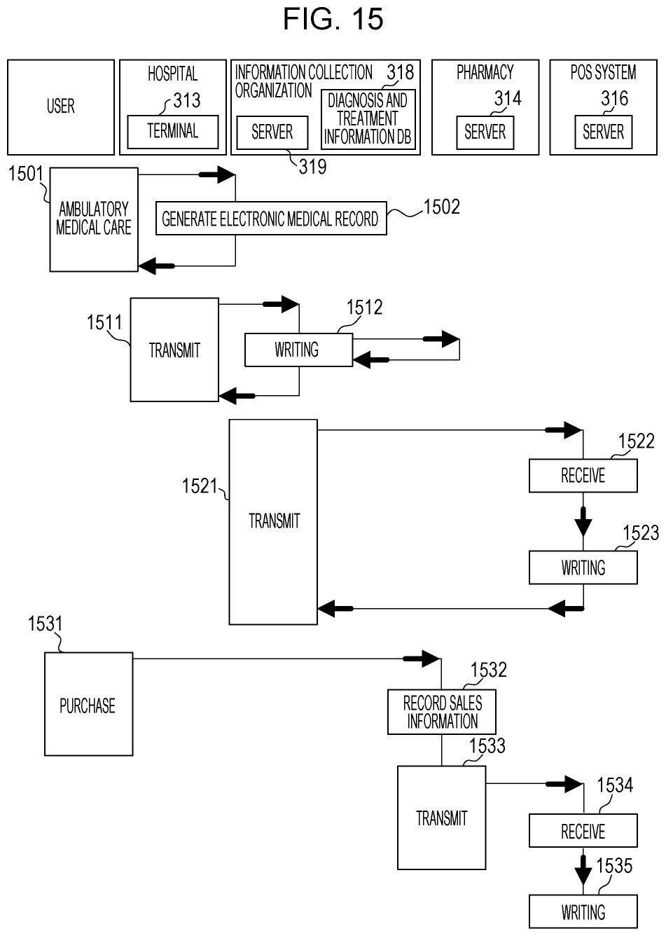

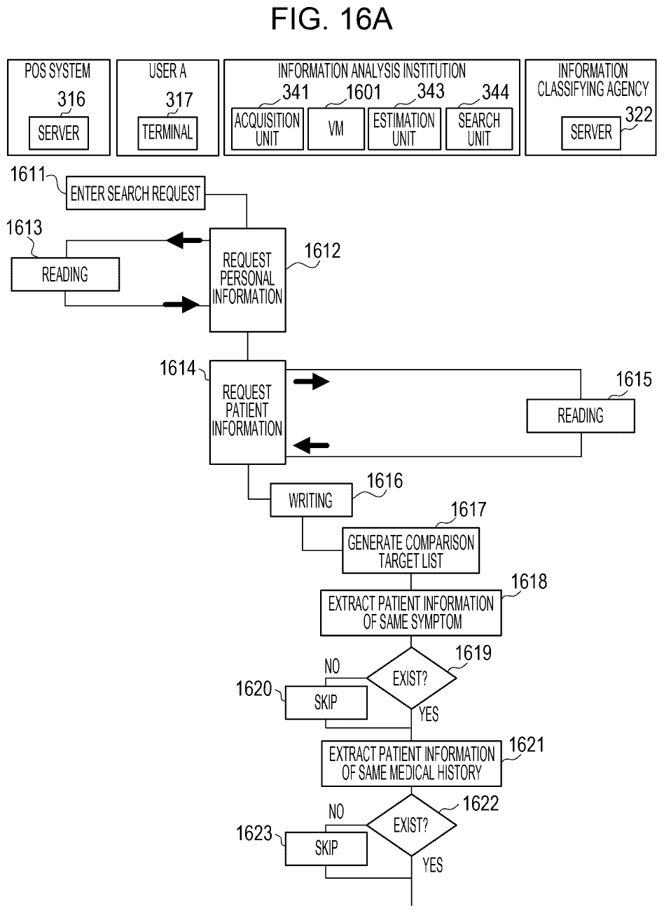

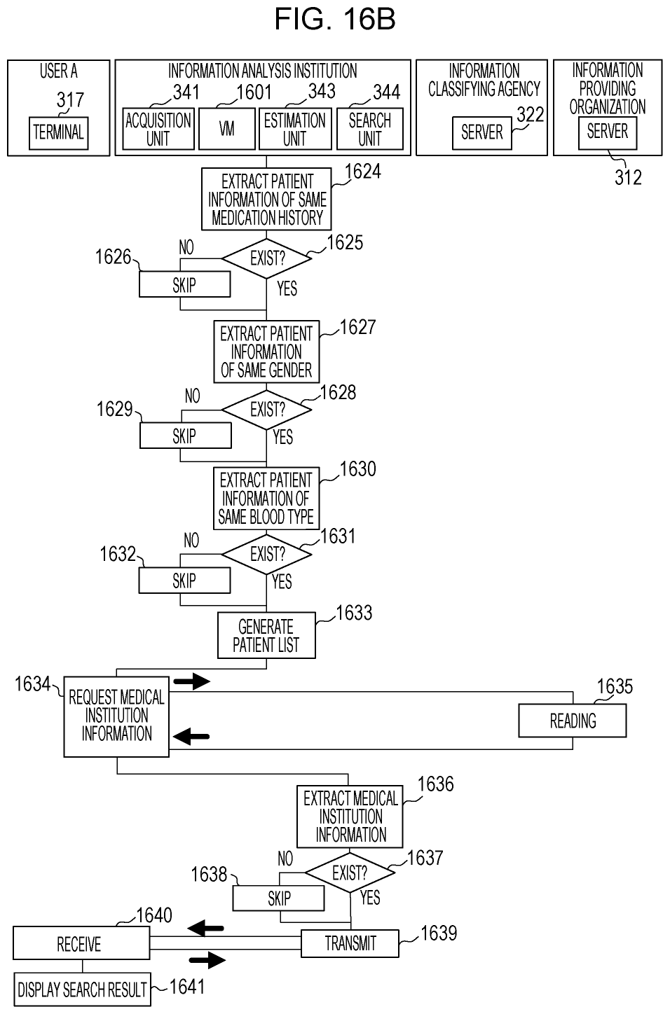

Next, the operation of the medical institution search system of FIG. 3 will be described in more detail with reference to FIGS. 15, 16A and 16B.

FIG. 15 illustrates an example of an information collection sequence. First, the user receives an ambulatory medical care (step 1501), and the terminal 313 of the hospital generates the electronic medical record 331 from the medical examination result input by the doctor (step 1502).

Next, the terminal 313 transmits the electronic medical record 331 to the server 319 of the information collection organization (step 1511), and the server 319 writes the received electronic medical record 331 into the diagnosis and treatment information DB 318 as diagnosis and treatment information of the user (step 1512).

Next, the server 319 transmits the diagnosis and treatment information to the server 316 of the PDS system (step 1521). The server 316 receives the diagnosis and treatment information (step 1522), and writes the received diagnosis and treatment information into the personal information DB 315 as the diagnosis and treatment information of the user (step 1523).

The user purchases the medicine at the pharmacy when the medicine is prescribed by the hospital doctor or when the user desires the medicine at his or her own discretion (step 1531). The server 314 of the pharmacy records the sales information input by the salesperson (step 1532), and transmits the sales information to the server 316 (step 1533). The server 316 receives the sales information (step 1534), and writes the received sales information into the personal information DB 315 as purchase information of the user (step 1535).

FIGS. 16A and 16B illustrate an example of a medical institution search sequence. First, the user A, who is the target patient, inputs a search request to the terminal 317, and the terminal 317 transmits the search request to the server 323 of the information analysis institution (step 1611).

The acquisition unit 341 of the server 323 extracts a user ID from the received search request, and transmits a personal information request including the user ID to the server 316 of the PDS system (step 1612).

The server 316 extracts the user ID from the received personal information request, and reads out the personal information of the user A corresponding to the user ID from the personal information DB 315 (step 1613). The server 316 transmits the read personal information to the server 323.

Next, the acquisition unit 341 transmits a patient information request to the server 322 of the information classifying agency (step 1614). The server 322 reads the integrated patient information from the integrated patient information DB 321, and transmits the integrated patient information to the server 323 (step 1615). The acquisition unit 341 writes the personal information and the integrated patient information in the storage unit 342 via a VM 1601 of the user A (step 1616).

Next, the estimation unit 343 generates a comparison target list from the search request and the personal information (step 1617), and extracts patient information including the symptom same as the symptom in the comparison target list from the integrated patient information (step 1618). At this time, the estimation unit 343 checks whether patient information including the same symptom is present in the integrated patient information (step 1619). When patient information including the same symptom is present (step 1619, YES), the estimation unit 343 extracts the patient information, and when no patient information including the same symptom is present (step 1619, NO), the extraction process is skipped (step 1620).

Next, the estimation unit 343 extracts patient information including the medical history same as any of the medical histories in the comparison target list from the extracted patient information or the remaining patient information (step 1621). At this time, the estimation unit 343 checks whether patient information including the same medical history is present (step 1622). When patient information including the same medical history is present (step 1622, YES), the estimation unit 343 extracts the patient information, and when no patient information including the same medical history is present (step 1622, NO), the extraction process is skipped (step 1623).

Next, the estimation unit 343 extracts patient information including the medication history same as any of the medical histories in the comparison target list from the extracted patient information or the remaining patient information (step 1624). At this time, the estimation unit 343 checks whether patient information including the same medication history is present (step 1625). When patient information including the same medication history is present (step 1625, YES), the estimation unit 343 extracts the patient information, and when no patient information including the same medication history is present (step 1625, NO), the extraction process is skipped (step 1626).

Next, the estimation unit 343 extracts patient information including the gender same as the gender in the comparison target list from the extracted patient information or the remaining patient information (step 1627). At this time, the estimation unit 343 checks whether patient information including the same gender is present (step 1628). When patient information including the same gender is present (step 1628, YES), the estimation unit 343 extracts the patient information, and when no patient information including the same gender is present (step 1628, NO), the extraction process is skipped (step 1629).

Next, the estimation unit 343 extracts patient information including the blood type same as the blood type in the comparison target list from the extracted patient information or the remaining patient information (step 1630). At this time, the estimation unit 343 checks whether patient information including the same blood type is present (step 1631). When patient information including the same blood type is present (step 1631, YES), the estimation unit 343 extracts the patient information, and when no patient information including the same blood type is present (step 1631, NO), the extraction process is skipped (step 1632).

Next, the estimation unit 343 generates a patient list including the estimated disease from the comparison target list and the patient information narrowed down by the process of steps 1618 to 1632 (step 1633).

Next, the acquisition unit 341 transmits a medical institution information request to the server 312 of the information providing organization (step 1634). The server 312 reads the medical institution information from the medical institution information DB 311, and transmits the medical institution information to the server 323 (step 1635).

Next, the search unit 344 extracts medical institution information including the estimated disease from information of the medical institution from which the patient has received a medical examination. At this time, the search unit 344 checks whether medical institution information including the estimated disease is present in the received medical institution information (step 1637). When medical institution information including the estimated disease is present (step 1637, YES), the search unit 344 extracts the patient information, and when no medical institution information including the estimated disease is present (step 1637, NO), the extraction process is skipped (step 1638).

When the medical institution information including the estimated disease is extracted, the search unit 344 generates a search result including the medical institution information, and when the extraction process is skipped, the search unit 344 generates a search result indicating no medical institution satisfying the search request is present.

The search unit 344 transmits the search result to the terminal 317 of the user A (step 1639), and the terminal 317 receives the search result (step 1640) and displays the received search result on the screen (step 1641).

The configuration of the information processing apparatus of FIG. 1 is merely an example, and some components may be omitted or changed depending on the application or conditions of the information processing apparatus.

The configuration of the medical institution search system of FIG. 3 is merely an example, and some components may be omitted or changed depending on the application or conditions of the medical institution search system. For example, the terminal 313 may be installed at another medical institution such as a clinic or a nursing home for the elderly.

The flowchart of FIG. 2 and the operation sequence of FIG. 15 to FIG. 16B are merely an example, and some processes may be omitted or changed depending on the configuration or conditions of the medical institution search system. For example, in the medical institution search sequence of FIG. 16A and FIG. 16B, when the integrated patient information is narrowed down using only the symptom in the comparison target list as a key, the process of steps 1621 to 1632 may be omitted.

The diagnosis and treatment information, the purchase information, the login information, the search request, and the comparison target list illustrated in FIGS. 4 to 8 are merely an example, and some items may be omitted or changed depending on the application or conditions of the medical institution search system. The narrowing-down result, the patient list, the medical institution information, and the search result illustrated in FIGS. 9 to 12 are merely an example, and these pieces of information change according to the information in the comparison target list. As the treatment result in FIGS. 11 and 12, the number of treated patients may be used instead of the cure rate.

The login screen illustrated in FIG. 13 and the search screen illustrated in FIG. 14 are merely an example, and some items may be omitted or changed depending on the application or conditions of the medical institution search system.

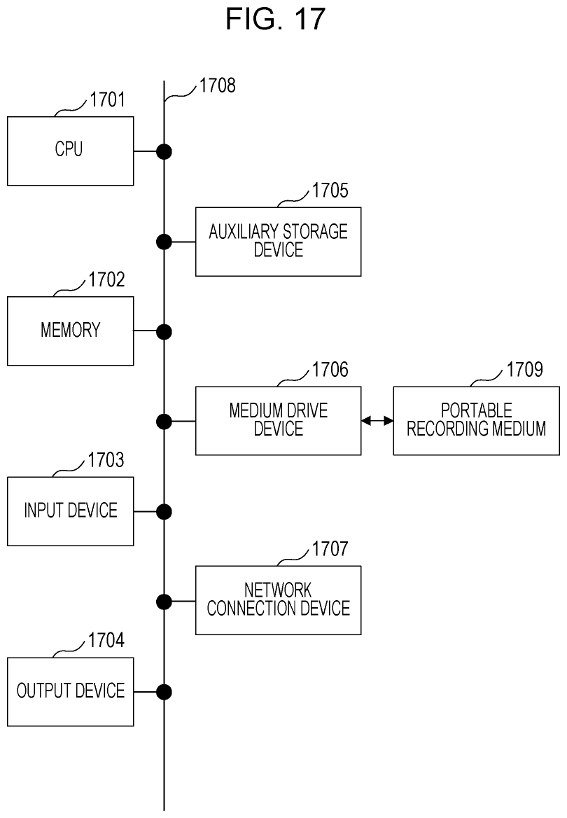

FIG. 17 illustrates an example of the configuration of hardware of the information processing apparatus used as the information processing apparatus 101 of FIG. 1 and the server 323 of FIG. 3. The information processing apparatus illustrated in FIG. 17 includes a central processing unit (CPU) 1701, a memory 1702, an input device 1703, an output device 1704, an auxiliary storage device 1705, a medium drive device 1706, and a network connection device 1707. These components are connected to each other by a bus 1708.

The memory 1702 is, for example, a semiconductor memory such as a read only memory (ROM), a random access memory (RAM), or a flash memory, and stores programs and data used for processing. The memory 1702 may be used as the storage unit 112 of FIG. 1 or the storage unit 342 of FIG. 3.

The CPU 1701 (processor) operates as the acquisition unit 111, the estimation unit 113, and the search unit 114 in FIG. 1 by executing a program using, for example, the memory 1702. The CPU 1701 operates as the acquisition unit 341, the estimation unit 343, and the search unit 344 in FIG. 3 by executing a program using the memory 1702.

The input device 1703 is, for example, a keyboard, a pointing device, or the like, and is used to input an instruction or information from an operator or a user. The output device 1704 is, for example, a display device, a printer, a speaker, or the like, and is used to output an inquiry or a processing result to the operator or the user.

The auxiliary storage device 1705 is, for example, a magnetic disk device, an optical disk device, a magneto-optical disk device, a tape device or the like. The auxiliary storage device 1705 may be a hard disk drive. The information processing apparatus may store programs and data in the auxiliary storage device 1705, and load them into the memory 1702 to use them. The auxiliary storage device 1705 may be used as the storage unit 112 of FIG. 1 or the storage unit 342 of FIG. 3.

The medium drive device 1706 drives a portable recording medium 1709 and accesses the recorded contents. The portable recording medium 1709 is a memory device, a flexible disk, an optical disk, a magneto-optical disk or the like. The portable recording medium 1709 may be a digital versatile disk (DVD), a compact disk read only memory (CD-ROM), a Universal Serial Bus (USB) memory, or the like. An operator or a user may store programs and data in this portable recording medium 1709, and load them into the memory 1702 to use them.

In this way, the computer readable recording medium that stores programs and data used for processing is physical (non-transitory) recording medium, such as the memory 1702, the auxiliary storage device 1705, or the portable recording medium 1709.

The network connection device 1707 is a communication interface circuit that is connected to a communication network such as a local area network (LAN) or a wide area network (WAN) and performs data conversion involved in communication. The information processing apparatus may receive programs and data from an external device via the network connection device 1707, and load them into the memory 1702 to use them.

The information processing apparatus may not include all the components illustrated in FIG. 17, and some of the components may be omitted depending on the application or conditions. For example, when it is not required to input an instruction or information from an operator or a user, the input device 1703 may be omitted, and when it is not required to output an inquiry or a processing result to an operator or a user, the output device 1704 may be omitted. When the portable recording medium 1709 is not used, the medium drive device 1706 may be omitted.

An information processing apparatus similar to that in FIG. 17 may be used as the server 312, the terminal 313, the server 314, the server 316, the terminal 317, the server 319, and the server 322 in FIG. 3.

Although one or more embodiments of the present disclosure have been described in detail, it should be understood that the various changes, substitutions, and alterations could be made hereto without departing from the spirit and scope of the disclosure.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An information processing apparatus comprising:
a processor configured to:
acquire, from a patient information storage device that stores patient information including a symptom of each of a plurality of patients and a medical history of each of the plurality of patients, the patient information of each of the plurality of patients,
extract first patient information including a symptom of a target patient from the patient information of each of the plurality of patients,
estimate a first disease corresponding to the symptom of the target patient based on a medical history included in the first patient information,
search for first medical institution information including the first disease, from medical institution information including identification information indicating each of a plurality of medical institutions and a disease that has been treated at each of the plurality of medical institutions, and
output first identification information included in the first medical institution information including the first disease; and
a memory coupled to the processor and configured to store the patient information of each of the plurality of patients.

2. The information processing apparatus of claim 1, wherein:
the patient information of each of the plurality of patients is classified patient information in which information identifying an individual is classified; and
the processor is configured to:
acquire, from a personal information storage device that stores diagnosis and treatment information of the target patient in a medical institution from which the target patient has received a medical examination before the symptom of the target patient occurs, the diagnosis and treatment information of the target patient, and
extract a medical history of the target patient from the diagnosis and treatment information of the target patient, extract second patient information including the symptom of the target patient and the medical history of the target patient from the patient information of each of the plurality of patients, and estimate the first disease corresponding to the symptom of the target patient based on the medical history included in the extracted second patient information.

3. The information processing apparatus of claim 2, wherein:
the patient information of each of the plurality of patients further includes a medication history of each of the plurality of patients;
the personal information storage device further stores purchase information indicating a medicine purchased by the target patient before the symptom of the target patient occurred; and
the processor is configured to:
acquire the purchase information from the personal information storage device, and
extract third patient information that includes the symptom of the target patient, the medical history of the target patient, and a medication history of a medicine indicated by the purchase information, from the patient information of each of the plurality of patients, and estimate the first disease corresponding to the symptom of the target patient based on the medical history included in the extracted third patient information.

4. The information processing apparatus of claim 3, wherein:
the patient information of each of the plurality of patients includes information of a plurality of items including a symptom, a medical history, and a medication history; and
the processor is configured to:
determine a degree of coincidence between first information of the plurality of items included in the first patient information that includes the symptom of the target patient, the medical history of the target patient, and the medication history of the medicine indicated by the purchase information, and second information of a plurality of corresponding items of the target patient, and
output a search result including the first identification information included in the first medical institution information including the estimated first disease and the degree of coincidence.

5. A medical institution search system comprising:
a patient information storage device including a first memory configured to store patient information including a symptom of each of a plurality of patients and a medical history of each of the plurality of patients; and
an information processing device including a second memory and a processor coupled to the second memory, the second memory being configured to store the patient information of each of the plurality of patients, wherein
the processor of the information processing device is configured to:
acquire, from the patient information storage device, the patient information of each of the plurality of patients,
extract first patient information including a symptom of a target patient from the patient information of each of the plurality of patients,
estimate a first disease corresponding to the symptom of the target patient based on a medical history included in the extracted first patient information,
search for first medical institution information including the estimated first disease, from medical institution information that includes identification information indicating each of a plurality of medical institutions and a disease that has been treated at each of the plurality of medical institutions, and
output first identification information included in the first medical institution information including the estimated first disease.

6. The medical institution search system of claim 5, further comprising:
a personal information storage device configured to store diagnosis and treatment information of the target patient in a medical institution from which the target patient has received a medical examination before the symptom of the target patient occurs, wherein
the patient information of each of the plurality of patients is classified patient information in which information identifying an individual is classified; and
the processor of the information processing device is configured to:
acquire, from the personal information storage device, the diagnosis and treatment information of the target patient, and
extract a medical history of the target patient from the diagnosis and treatment information of the target patient, extract second patient information including the symptom of the target patient and the medical history of the target patient from the patient information of each of the plurality of patients, and estimate the first disease corresponding to the symptom of the target patient based on the medical history included in the extracted second patient information.

7. The medical institution search system of claim 5, wherein:
the patient information of each of the plurality of patients further includes a medication history of each of the plurality of patients;
the personal information storage device further stores purchase information indicating a medicine purchased by the target patient before the symptom of the target patient occurred; and
the processor of the information processing device is configured to:
acquire the purchase information from the personal information storage device, and
extract third patient information that includes the symptom of the target patient, the medical history of the target patient, and a medication history of a medicine indicated by the purchase information, from the patient information of each of the plurality of patients, and estimate the first disease corresponding to the symptom of the target patient based on the medical history included in the extracted third patient information.

8. The medical institution search system of claim 5, wherein:
the patient information of each of the plurality of patients includes information of a plurality of items including a symptom, a medical history, and a medication history; and
the processor of the information processing device is configured to:
determine a degree of coincidence between first information of the plurality of items included in the first patient information that includes the symptom of the target patient, the medical history of the target patient, and the medication history of the medicine indicated by the purchase information, and second information of a plurality of corresponding items of the target patient, and
output a search result including the first identification information included in the first medical institution information including the estimated disease and the degree of coincidence.

9. A non-transitory, computer-readable recording medium having stored therein a program for causing a computer to execute a process comprising:
acquiring, from a patient information storage device that stores patient information including a symptom of each of a plurality of patients and a medical history of each of the plurality of patients, the patient information of each of the plurality of patients;
storing the patient information of each of the plurality of patients in a memory;
extracting first patient information including a symptom of a target patient from the patient information of each of the plurality of patients;
estimating a first disease corresponding to the symptom of the target patient based on a medical history included in the extracted first patient information;
searching for first medical institution information including the first disease from medical institution information including identification information indicating each of a plurality of medical institutions and a disease that has been treated at each of the plurality of medical institutions; and
outputting first identification information included in the first medical institution information including the estimated disease.

10. The non-transitory, computer-readable recording medium of claim 9, wherein:
the patient information of each of the plurality of patients is classified patient information in which information identifying an individual is classified; and
the process further comprises:
acquiring, from a personal information storage device that stores diagnosis and treatment information of the target patient in a medical institution from which the target patient has received a medical examination before the symptom of the target patient occurs, diagnosis and treatment information of the target patient, and
extracting a medical history of the target patient from the diagnosis and treatment information of the target patient, extract second patient information including the symptom of the target patient and the medical history of the target patient from the patient information of each of the plurality of patients, and estimate the first disease corresponding to the symptom of the target patient based on the medical history included in the extracted second patient information.

11. The non-transitory, computer-readable recording medium of claim 10, wherein:
the patient information of each of the plurality of patients further includes a medication history of each of the plurality of patients;
the personal information storage device further stores purchase information indicating a medicine purchased by the target patient before the symptom of the target patient occurred; and
the process further comprises:
acquiring the purchase information from the personal information storage device, and
extracting third patient information that includes the symptom of the target patient, the medical history of the target patient, and a medication history of a medicine indicated by the purchase information, from the patient information of each of the plurality of patients, and estimate the first disease corresponding to the symptom of the target patient based on the medical history included in the extracted third patient information.

12. The non-transitory, computer-readable recording medium of claim 11, wherein:
the patient information of each of the plurality of patients includes information of a plurality of items including a symptom, a medical history, and a medication history; and
the process further comprises:
determining a degree of coincidence between first information of the plurality of items included in the first patient information that includes the symptom of the target patient, the medical history of the target patient, and the medication history of the medicine indicated by the purchase information, and second information of a plurality of corresponding items of the target patient, and
outputting a search result including the first identification information included in the first medical institution information including the estimated first disease and the degree of coincidence.
